(19) United States
(12) Patent Application Publication
(10) Pub. No.: US 2019/0239945 A1
(43) Pub. Date: Aug. 8, 2019

(54) CATHETER HANDLE WITH RING COLOR INDICATORS

(71) Applicant: BIOSENSE WEBSTER (ISRAEL) LTD., Yokneam (IL)

(72) Inventors: Eliyahu Ravuna, Kiryat Ata (IL); Lior Botzer, Timrat (IL); Shmuel Auerbach, Kerem Maharal (IL); Aharon Turgeman, Rehovot (IL)

(21) Appl. No.: 15/890,023

(22) Filed: Feb. 6, 2018

(51) Int. Cl. A61B 18/14

(52) U.S. Cl. CPC A61B 18/1492; A61B 5/6852

(57) ABSTRACT

A medical instrument includes a handle and a ring-shaped visual indicator. The ring-shaped visual indicator surrounds a perimeter of the handle and is configured to illuminate a visual indication in response to being driven by a driver-signal.

### FIELD OF THE INVENTION

The present invention relates generally to medical instruments, and particularly to user-interfaces of catheters and other intra-body probes.

### BACKGROUND OF THE INVENTION

User interfaces are commonly incorporated in many sensing and treatment systems, including catheter-based systems. For example, U.S. Patent Application Publication 2011/0172659 describes an ablation device that includes a handle assembly including a distal end, an ablation probe extending distally from the distal end of the handle assembly, and a user interface disposed at the handle assembly. The ablation probe is operably coupled to the user interface. The user interface includes a controller unit, a power on/off switch operably coupled to the controller unit, and at least one detector capable of generating an electrical signal. The controller is in communication with the at least one detector and configured to override operation of the power on/off switch in response to the electrical signal generated by the at least one detector.

As another example, U.S. Pat. No. 9,289,258 describes a medical system for delivering treatment or therapy to a patient, which has a kill switch for interrupting the delivery. The kill switch, which can disrupt the delivery directly or can cause an error message to be generated that disrupts the delivery, can be activated by the operator or remotely. In an ablation catheter system, a kill switch mechanism immediately and abruptly terminates delivery of ablation treatment or therapy. In an embodiment, the kill switch comprises a button that is configured so that when an ablation catheter is “hot” or energized, electricity travels through the catheter handle to light the button or to activate a light, LED, or visual or audible alert in the handle, so that the operator is aware that the catheter is delivering ablation therapy. The invention described herein could utilize a myriad of buttons, controls, or switches with indicators that function and/or provide information including LEDs, the flow of electricity notification, audible tones, etc.

U.S. Patent Application Publication 2014/0275991 describes systems for detecting when catheter electrodes enter into and exit from an introducer. In one form, a system detects a relative position of a catheter (comprising a marker band and an electrode) and an introducer (comprising a proximity sensor adapted to sense the marker band), while the catheter and introducer are in a human body. The system may comprise an electronic control unit to analyze signals from the catheter and/or the introducer, to determine whether the catheter electrode is within the introducer, and to disregard data collected from the electrode when that electrode is in the introducer. The sensor may be on the catheter and the sensed element may be on the introducer. The sensed element may comprise one or several marker bands. In an embodiment, an indicator light on the catheter's handle could be red until a marker band is detected by the proximity sensor and then go out or turn green.

U.S. Patent Application Publication 2014/0276760 describes an ablation apparatus that includes a flexible probe adapted for insertion into a heart of a living subject. The probe has a distally disposed ablation electrode to be brought into contact with a target tissue in the heart, and has facilities for measuring contact force with the target tissue. The apparatus includes a transmitter, operative to transmit an indication of the contact force to a wearable device having an actuator operative to haptically stimulate the operator responsively to the indication.

U.S. Patent Application Publication 2012/0184955 describes a system for remotely controlling the positioning within the body of a patient of an elongated medical device comprising a control handle and a handle controller. The handle controller can be hard-wired or wireless, the handle controller providing HAPTIC (i.e., feel) feedback through a resistive, vibratory, sound, and or color-coded LED mechanism. In an embodiment, a remote controller handle is equipped with an audio and visual feedback mechanism, namely a speaker and a light. The visual feedback mechanism may light up in response to a variety of operating conditions or circumstances.

### SUMMARY OF THE INVENTION

An embodiment of the present invention provides a medical instrument including a handle and a ring-shaped visual indicator. The ring-shaped visual indicator surrounds a perimeter of the handle and is configured to illuminate a visual indication in response to being driven by a driver-signal.

In some embodiments, the medical instrument includes a processor configured to receive a signal transmitted by a sensor, store a pre-set limit, and based on the received signal and on the pre-set limit, generate the driver-signal to drive the ring-shaped visual indicator.

In some embodiments, the processor is configured to receive the pre-set limit via a user-interface.

In an embodiment, the ring-shaped visual indicator is configured to illuminate in a color selected from multiple colors responsively to the driver-signal.

In another embodiment, the ring-shaped visual indicator is configured to illuminate in a selected shade of a same color, the shade selected responsively to the driver-signal.

In an embodiment, the ring-shaped visual indicator is configured to illuminate in an illumination intensity selected responsively to the driver-signal.

In another embodiment, the ring-shaped visual indicator is configured to blink responsively to the driver-signal.

In an embodiment, the ring-shaped visual indicator is configured to blink in a blinking pattern selected responsively to the driver-signal.

There is additionally provided, in accordance with an embodiment of the present invention, a method including driving a ring-shaped visual indicator, which surrounds a perimeter of a handle of a medical instrument, with a driver-signal. A visual indication is illuminated by the ring-shaped visual indicator responsively to the driver-signal.

There is also provided, in accordance with an embodiment of the present invention, a medical instrument that includes a handle and a visual indicator. The visual indicator surrounds at least part of a perimeter of the handle and is configured to illuminate a visual indication in response to being driven by a driver-signal, wherein the visual indicator is viewable by a user manipulating the medical instrument irrespective of a rotation angle of the handle.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based cardiac sensing and ablation system, in accordance with an embodiment of the present invention;

FIG. 2 is schematic, pictorial illustration of the catheter handle seen in FIG. 1, in accordance with an embodiment of the present invention;

FIGS. 3A and 3B are schematic, pictorial illustrations of ring color indications on a catheter handle, in accordance with an embodiment of the present invention; and

FIG. 4 is a flow chart schematically illustrating a method for providing ring indication, in accordance with an embodiment of the present invention.

### DETAILED DESCRIPTION OF EMBODIMENTS

### Overview

Embodiments of the present invention that are described herein provide one or more ring shaped color indicators that are fitted at a catheter handle, and ring color indication methods. The one or more ring indicators surround perimeters of the handle and are thus easily observable from any angle by the physician that holds and manipulates the handle.

The ring indicators are configured to illuminate visual indications responsively to being driven by one or more respective driver-signals, as described below. The disclosed color indication methods assist in maintaining the physician awareness of critical information in real-time, since the one or more indicators display the indications within a field of view where the physician's visual attention is usually anyway mostly directed at.

In some embodiments, various sensors are either coupled to the distal end of the catheter or located elsewhere. The sensors transmit sensory-signals to a processor in response to physical inputs the sensors detect. The physical inputs may vary, and include, for example, patient breath, patient and room temperatures, pressure, force, tissue proximity, heart rate, and electrical ablative power. The processor analyzes the sensory-signals and, responsively and accordingly to these and to pre-set stored limits, it generates, via an interface, corresponding driver-signals. The driver-signals drive the one or more ring visual indicators, which may comprise, for example, LED, OLED, Backlit LCD or E-Ink.

As noted above, driver-signals may depend on pre-set limits. In some embodiments, one or more limit values are stored in, or coded into, the processor, and/or set into the processor via a user-interface. Such limits may include, for example, a minimum value, a maximum value and an allowed range. When the processor analyzes the sensory-signals, it compares the sensory-signals with the pre-set limits. The resulting driver-signals depend on the outcome of that comparison. For example, in an embodiment, the ring color indicators (that provide indications of physical inputs) are configured to receive driver-signals and to illuminate different colors responsively to whether the physical input is within an allowed range or outside the allowed range.

In an embodiment, a ring indicator provides visual indication by illuminating multiple colors responsively to one or more respective driver-signals. The ring indicators can provide visual indication by blinking responsively to one or more driver-signals. The blinking feature may be useful to alert the physician. Alternatively or additionally, the ring indicators may change illumination intensity responsively to the driver-signals.

In some embodiments, color indicators are fitted at a catheter handle in such a way that a physician will be able to view them regardless of an orientation-angle by which he may rotate the handle, while performing a procedure using the catheter. Numerous shapes of indicators may be realized for this purpose, such as the above noted full rings, structured rings that may include gaps, and partial-ring indicators that cover most but not entirely all of the perimeter, to mention only a few options that meet the above requirement.

The disclosed ring color indicators and ring color indication methods assist the physician in receiving critical feedback in real-time during an invasive procedure while remaining in focus on the patient and on his own actions. Specifically, the ring color indicators may assist in minimizing any adverse effect by alarming the physician that he is about to exceed certain limit. The ring indicators and ring indication methods may further free the physician's attention to priority actions he must take throughout a procedure.

### System Description

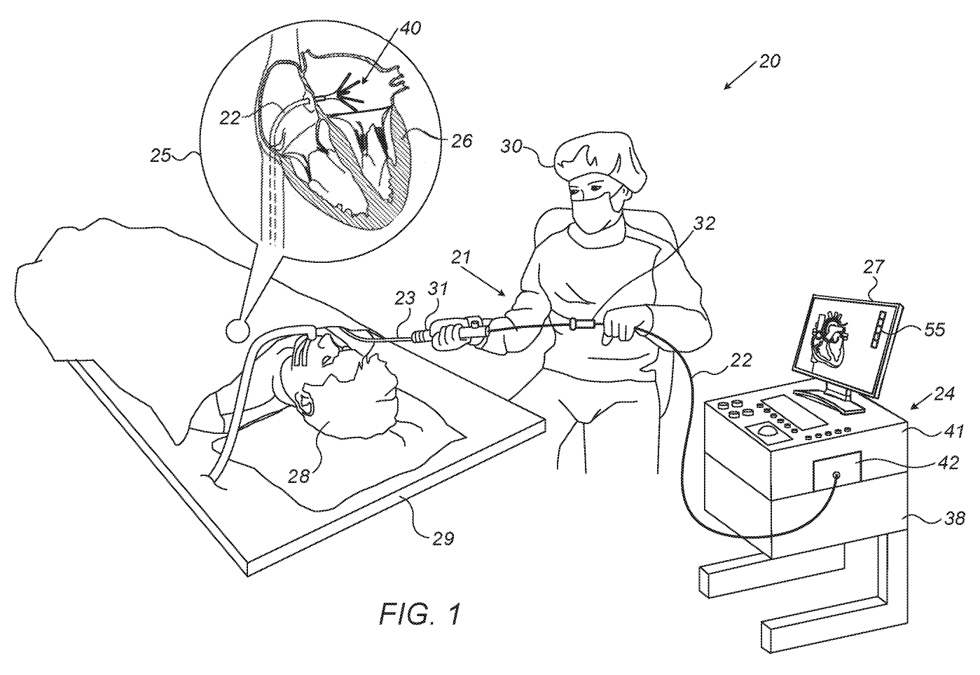

FIG. 1 is a schematic, pictorial illustration of a catheter-based cardiac sensing and ablation system 20, in accordance with an embodiment of the present invention. System 20 comprises a catheter 21, having a catheter handle 31. The distal end of a shaft 22 of the catheter is inserted through a sheath 23 into a heart 26 of a patient 28 lying on a table 29. The proximal end of catheter 21 is connected to a console 24. In the embodiment described herein, catheter 21 may be used for any suitable therapeutic and/or diagnostic purposes, such as pressure sensing, strain sensing, electrical sensing, and/or an ablation of tissue in heart 26. In the description herein after, by way of example, the distal end of shaft 22 is fitted with a device 40, shown in an inset 25. Device 40 includes contact force sensors and Radiofrequency (RF) ablation-electrodes. Device 40 is configured to measure contact force with tissue of heart 26, and to ablate tissue.

Physician 30 navigates the distal end of shaft 22 to a target location in heart 26 by holding catheter handle 31 with his right hand, and manipulating shaft 22 using a manipulator 32 near the proximal end of the catheter and/or deflection from the sheath 23. During the insertion of shaft 22, device 40 is maintained in a collapsed configuration by sheath 23. By containing device 40 in a collapsed configuration, sheath 23 also serves to minimize vascular trauma along the way to target location.

Console 24 comprises a processor 41, typically a general-purpose computer, with suitable front end and interface circuits 38 for receiving signals from catheter 21, such as contact-force signals, as well as for applying treatment via catheter 21 in heart 26 and for controlling the other components of system 20. In some embodiments, processor 41 may be configured to receive multiple sensed-signals, such as sensed-signals from the contact force sensors fitted at device 40, and calculate from these sensed-signals, and depending on pre-set limits processor 41 stores, driver-signals, such as driver-signals related to the extent to which device 40 is in contact with surrounding tissue of heart 26, relative to pre-set limits. In some embodiments, an output interface 42 drives by outputting driver-signals, ring color indicators located on catheter handle 31.

The indications may be presented to physician 30 also on a monitor 27, e.g., in a graphical form of a bar diagram 55. As seen, physician 30 would have to regularly turn his view away observe the monitor. Even if the monitor is can be moved in front of him, physician 30 would still have to refocus his view back and forth throughout the invasive procedure, between following his own actions with the catheter and on the patient, and for capturing information displayed in the background.

Processor 41 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

The example configuration shown in FIG. 1 is chosen purely for the sake of conceptual clarity. The disclosed techniques may similarly be applied using other system components and settings. For example, system 20 may comprise other components and perform non-cardiac catheterization treatments.

### Catheter Handle with Ring Color Indicators

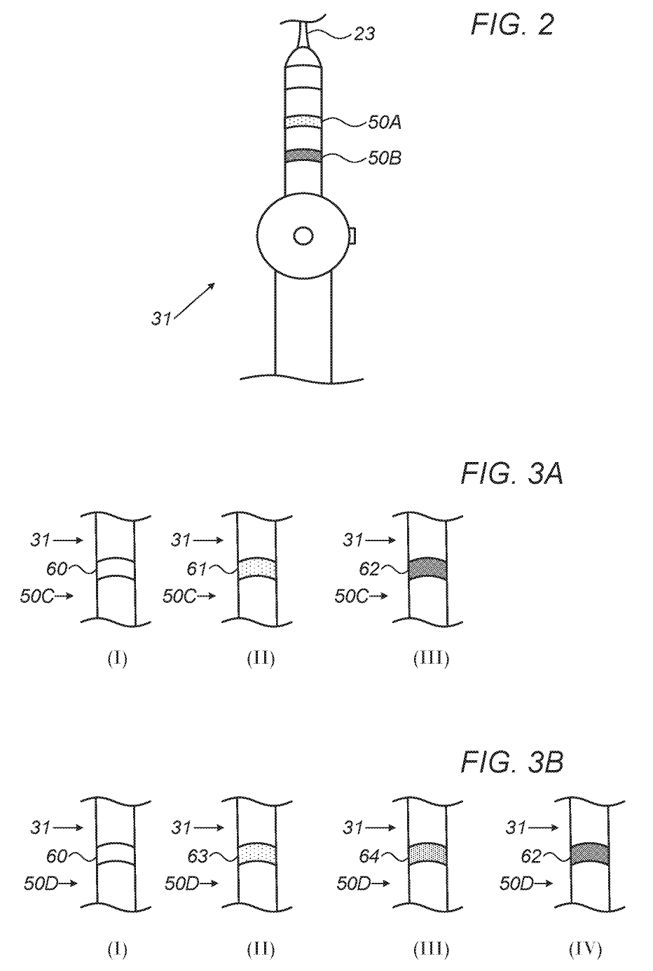

FIG. 2 is schematic, pictorial illustration of catheter handle 31 seen in FIG. 1, in accordance with an embodiment of the present invention. In the configuration exemplified in FIG. 2, handle 31 comprises two ring shaped color indicators, an indicator 50A and an indicator 50B. The indicators are close to a distal end of handle 31, proximally to end location of sheath 23. In some embodiments, indicators 50A and 50B provide visual feedback to physician 30 responsively to respective driver-signals they receive (e.g., in the form of a voltages directed by a commands processor 41 provides, causing indicators 50A and/or 50B to illuminate with certain colors).

In some embodiments, indicators 50A and 50B provide visual feedback to physician 30 on the performance of various devices involved an invasive treatment that the physician performs. For example, indicator 50A may provide color indication of the extent to which device 40 is in contact with surrounding tissue of heart 26, and indicator 50B may provide color indication of tissue temperature during ablation.

In an embodiment, indicators 50A and 50B are shaped in the form of illuminating rings surrounding the perimeter of catheter handle 31. The ring shape ensures physician 30 can observe the required visual indication at various situations, for example, no matter how much and in which direction catheter handle 31 is turned around.

The example illustration shown in FIG. 2 is chosen purely for the sake of conceptual clarity. Additional ring indicators may be included in handle 31, for example to provide visual indication of the RF intensity, of the heart rate, and more. Generally, handle 31 may comprise any suitable number of ring indicators, or even a single ring indicator. Each ring indicators may comprise a full or partial ring. Each ring indication may visualize any suitable value or event.

In some embodiments, the ring indicators are configured to illuminate multiple colors corresponding one or more indications and responsively to one or more respective driver-signals. In an embodiment, ring indicators such as 50A and 50B are configured to blink responsively to the one or more driver-signals. The pattern of blinking may depend on the type and/or values of respective driver-signals. In another embodiment ring indicators such as 50A and 50B are configured to change illumination intensity responsively to the one or more driver-signals.

Other forms of indicators are possible, for example a set of bars, dot matrix display, and more, which may surround the perimeter of the handle. Half-ring indicators may be used. The light illumination technology may comprise, for example, LED, OLED, Backlit LCD or E-Ink, or any other.

In an embodiment, the physician may choose which real-time parameter is tied to which ring display of one or more fitted on the handle. In another embodiment, the processor may offer a list of possible real-time parameters to the physician, so that the physician could indicate his or her preferences.

The color illuminated by a given ring indicator may be calculated by an electronic circuit located in the catheter handle, or by a processor that may directly or indirectly communicate with the catheter, where the processor is optionally located in a workstation, a dongle, a user-interface, or elsewhere.

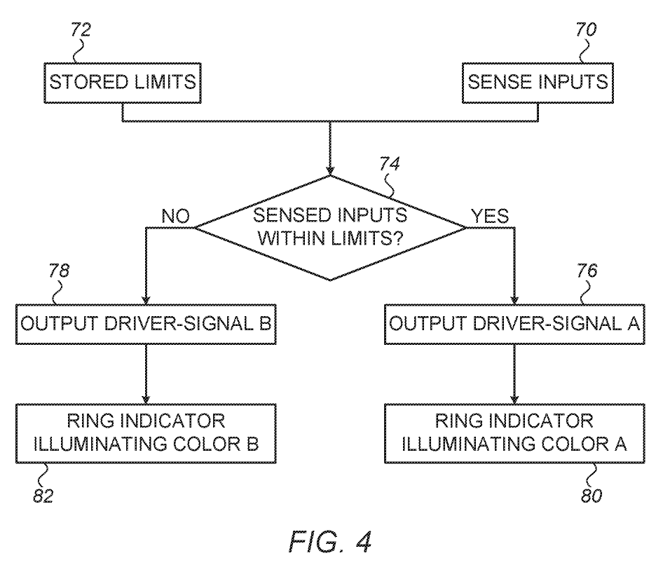

FIGS. 3A and 3B are schematic, pictorial illustrations of ring color indications on a catheter handle, in accordance with an embodiment of the present invention.

In an embodiment, physician 30 pre-sets and stores in processor 41 limits and/or ranges for different parameters to be indicated during procedure. The parameters may be set in advance by physician 30 so as for the physician to receive distinct visual indications when one of these limits is crossed.

FIG. 3A, for example, shows a ring color indicator 50C that indicates minimal and maximal values for the extent of the mechanical force that device 40 is exerting over tissue. In the above example, during the procedure, indicator 50C illuminates a white light 60 if the real-time measured force is less than certain low limit value F1. Visual indicator 50C illuminates a green light 61 if the real-time measured force is between F1 and a high limit value F2, and illuminates a red light 62 if the real-time force is greater than the high limit value F2.

In another example, FIG. 3B shows a ring color indicator 50D that indicates of a three-range setting of an ablation index vs. certain limiting parameters that the physician may adjust. In the example by FIG. 3B, during the procedure, indicator 50D illuminates a white light 60 if the real-time ablation index is less than certain low limit value A1. Visual indicator 50D illuminates a clear pink light 63 if the real-time ablation index is between A1 and a higher limit value A2, a dark pink light 64 if the real-time ablation index is between A2 and yet a higher limit value A3, and a red light 62 if the ablation index is greater than the highest limit value A3.

The example illustrations shown in FIGS. 3A and 3B are chosen purely for the sake of conceptual clarity. Other numbers of pre-set limits may be possible per each ring color indicator. In an embodiment, an indicator is configured to illuminate multiple colors or shades of a color corresponding to values of an ablation index. Other modes of indication are possible, such as blinking, blinking combined with audio alarming, to name just a few examples.

FIG. 4 is a flow chart schematically illustrating a method for providing ring indication, in accordance with an embodiment of the present invention. The process begins with one or more sensors transmitting one or more signals responsively to one or more respective detected physical inputs, at a sensing step 70. A processor (e.g., processor 41 of FIG. 1) then checks the sensed signal in comparison with pre-set stored limits, at a checking step 72.

If the sensed signal is within the pre-set limits, the processor decides accordingly to output a driver-signal A, at a decision step 74, and correspondingly outputs, via output interface 42, the driver-signal A, at an outputting step 76. If the sensed signal is above the pre-set limits, then the processor decides accordingly to output a driver-signal B, at step 74, and correspondingly outputs, via output interface 42, the driver-signal B, at outputting step 78.

Either driver-signal A or driver-signal B are transmitted to a ring indicator which illuminates correspondingly, in color A in an indicating step 80, or in color B, at an indicating step 82, respectively.

The example illustration shown in FIG. 4 is chosen purely for the sake of conceptual clarity. The number of limits can be one or more, and the color indication may include blinking or any other type of indication.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
a handle; and
a ring-shaped visual indicator, which surrounds a perimeter of the handle and is configured to illuminate a visual indication in response to being driven by a driver-signal.

2. The medical instrument according to claim 1, and comprising a processor configured to:
receive a signal transmitted by a sensor;
store a pre-set limit; and
based on the received signal and on the pre-set limit, generate the driver-signal to drive the ring-shaped visual indicator.

3. The medical instrument according to claim 2, wherein the processor is configured to receive the pre-set limit via a user-interface.

4. The medical instrument according to claim 1, wherein the ring-shaped visual indicator is configured to illuminate in a color selected from multiple colors responsively to the driver-signal.

5. The medical instrument according to claim 1, wherein the ring-shaped visual indicator is configured to illuminate in a selected shade of a same color, the shade selected responsively to the driver-signal.

6. The medical instrument according to claim 1, wherein the ring-shaped visual indicator is configured to illuminate in an illumination intensity selected responsively to the driver-signal.

7. The medical instrument according to claim 1, wherein the ring-shaped visual indicator is configured to blink responsively to the driver-signal.

8. The medical instrument according to claim 7, wherein the ring-shaped visual indicator is configured to blink in a blinking pattern selected responsively to the driver-signal.

9. A method, comprising:
driving a ring-shaped visual indicator, which surrounds a perimeter of a handle of a medical instrument, with a driver-signal; and
illuminating a visual indication by the ring-shaped visual indicator responsively to the driver-signal.

10. The method according to claim 9, and comprising:
receiving, in a processor, a signal transmitted by a sensor;
storing a pre-set limit; and
based on the received signal and on the pre-set limit, generating the driver-signal to drive the ring-shaped visual indicator.

11. The method according to claim 10, and comprising receiving the pre-set limit in the processor via a user-interface.

12. The method according to claim 9, wherein illuminating the visual indication comprises illuminating in a color selected from multiple colors responsively to the driver-signal.

13. The method according to claim 9, wherein illuminating the visual indication comprises illuminating in a selected shade of a same color, the shade selected responsively to the driver-signal.

14. The method according to claim 9, wherein illuminating the visual indication comprises illuminating in an illumination intensity selected responsively to the driver-signal.

15. The method according to claim 9, wherein illuminating the visual indication comprises blinking responsively to the driver-signal.

16. The method according to claim 15, wherein illuminating the visual indication comprises blinking in a blinking pattern selected responsively to the driver-signal.

17. A medical instrument, comprising:
a handle; and
a visual indicator, which surrounds at least part of a perimeter of the handle and is configured to illuminate a visual indication in response to being driven by a driver-signal, wherein the visual indicator is viewable by a user manipulating the medical instrument irrespective of a rotation angle of the handle.
